(19) United States
(12) Patent Application Publication
(10) Pub. No.: US 2023/0390068 A1
(43) Pub. Date: Dec. 7, 2023

(54) DEVICES AND METHODS FOR SEMI-RIGID BONE FIXATION

(71) Applicant: DePuy Synthes Products, Inc., Raynham, MA (US)

(72) Inventors: Todd J. KENT, Philadelphia, PA (US); Edward KUREK, Lansdale, PA (US)

(73) Assignee: DePuy Synthes Products, Inc., Raynham, MA (US)

(21) Appl. No.: 17/831,565

(22) Filed: Jun. 3, 2022

(51) Int. Cl. A61F 2/30; A61B 17/80

(52) U.S. Cl. CPC A61F 2/30; A61B 17/8019; A61F 2002/30672; A61F 2002/30622; A61F 2002/30462; A61F 2002/30476; A61F 2002/3085

(57) ABSTRACT

An example apparatus is provided for placing a first anchor in a first bone, and a locking assembly in a second bone, the first anchor and locking assembly being connected by a flexible segment that provides substantial tensile stabilization but offers little or no resistance to bending, rotation, or shear motion of the first anchor and locking assembly with respect to each other. The first anchor can include a proximal end and a distal end configured for insertion into a first hole in the first bone. The locking assembly can include a proximal end and a distal end configured for insertion into a second hole in the second bone. The flexible segment can be configured to adjust a distance between the first and second bones, while the locking assembly releasably engages the flexible segment to fix the distance between the first and second bones.

### FIELD

The present invention generally relates to devices and methods for semi-rigid fixation of bones. More specifically, certain embodiments relate to systems and methods for fixation of the distal tibia and distal fibula following an injury to the corresponding syndesmotic joint.

### BACKGROUND

A syndesmotic injury results when a traumatic injury damages the ligaments that span the gap between the distal tibia and fibula. This can be the result of a high ankle sprain, with no fracture of the fibula, or can also accompany a fibular fracture in a Weber B or Weber C fracture.

A surgeon can determine the presence of a syndesmotic injury by direct visualization of the joint or through radiographic imaging while positioning the ankle in a mortise view orientation. In either case, loads are applied to the joint in either a direct lateral load applied to the fibula or by applying an external rotation load to the foot. While the load is being applied, the relative distance between the fibula and the tibia, the fibula and the talus, and the tibia and the talus are observed to determine the level of damage sustained by the ligaments that typically hold the syndesmotic joint together.

If a syndesmotic injury is found to be present, the typical treatment involves stabilizing the fibula and tibia with respect to each other in the proper orientation and holding them there throughout the soft tissue healing period to allow the ligaments to re-attach and heal. In the event of a syndesmotic injury with a corresponding fibula fracture, this is done while also stabilizing the fibular fracture, which is usually accomplished with a small fracture plate on the lateral side of the fibula. Traditionally the method of stabilization has been to place one or more cortical screws across the syndesmosis, with the head against the lateral face of the fibula and the tip of the screw being in the middle of the tibia or in the medial cortex of the tibia.

This form of treatment provides very rigid fixation, allowing the ligaments to heal, but makes return to weight-bearing more difficult. During a standard gait, the ligaments hold the distance between the tibia and fibula fairly constant, but allow a small amount of shear motion and rotation of the fibula with respect to the tibia. The presence of the fixation screws prevents this motion and can cause discomfort and limited flexibility of the ankle joint. Typically, the surgeon prescribes a secondary surgery to remove the screws once the ligaments have healed. In some cases, a surgeon may simply recommend a return to weight-bearing when the ligaments have healed and, after a period of time of loading the screws, they will experience a fatigue failure and normal anatomical motion will be restored.

To address these rigidity issues, some methods of stabilization have been developed to include a flexible internal segment connected by a first anchor on the lateral side of the fibula and a second anchor on the medial side of the tibia. These methods, however, require a through or bore hole through the medial wall of the tibia, which not only provides an additional point of necessary recovery, but also requires a physician to access a patient from multiple sides and angles during treatment.

Accordingly, alternative apparatus and methods for providing semi-rigid fixation of the distal tibia and fibula following a syndesmotic injury would be useful.

### SUMMARY

The present invention is directed to apparatus and methods for stabilizing a joint between two bones, e.g., the tibia and fibula, during the soft tissue healing period following a traumatic injury.

In an exemplary application, the apparatus and methods herein may be configured to provide substantially rigid tensile fixation between the tibia and fibula while allowing the small amount of shear and rotational motion required for a standard gait. This may make it possible for patients to return to weight-bearing earlier, which may improve clinical outcomes, and/or may also reduce the number of follow-up hardware-removal surgeries.

An example apparatus is provided for placing a first anchor in a first bone, such as a tibia, and a locking assembly in a second bone, such as a fibula, the first anchor and locking assembly being connected by a flexible segment that provides substantial tensile stabilization but offers little or no resistance to bending, rotation, or shear motion of the first anchor and locking assembly with respect to each other. The first anchor can include a proximal end and a distal end configured for insertion (e.g., via threading, pushing, etc.) into a first hole in the first bone. The locking assembly can include a proximal end and a distal end configured for insertion into a second hole in the second bone. The flexible segment (e.g., including one or more suture threads) can be configured to extend between the first anchor and the locking assembly, and to adjust a distance between the first and second bones. The locking assembly can be configured to releasably engage the flexible segment to fix the distance between the first and second bones.

The first anchor can pass through the second hole and can be inserted (e.g., threaded or pushed) within the first bone in the first hole from its distal end to its proximal end. The first hole can be disposed on a first side of the first bone, and the first anchor can be configured such that there is a certain distance between the distal end of the first anchor and a second side of the first bone. That is, there may be no second hole formed in the first bone. Instead, the first anchor can be configured such that it can be inserted into, but not through, the first bone.

The locking assembly can be coupled to a bone plate coupled to a first side of the second bone.

The flexible segment can extend between and/or beyond the distal end of the locking assembly and the proximal end of the first anchor.

The locking assembly can include one or more openings configured to receive the flexible segment, and can anchor a proximal end of the flexible segment to a first side of the second bone.

The locking assembly can include a body and a locking insert. The body can include a proximal end, a distal end, and an internal cavity. The locking insert can be moveable from a first position to a second position within the internal cavity of the body, and can transition from the first position to the second position to lock the flexible segment at a certain length.

In another example apparatus, the locking assembly can include a locking insert and a button, which can include a first opening and one or more second openings. The first opening can accept a distal end of the locking insert, while the second openings can accept one or more proximal ends of one or more suture threads of the flexible segment. The locking assembly can lock the flexible segment at a certain length when the proximal ends of the suture threads are pulled in a proximal direction through the second openings.

The locking insert of the locking assembly can include a cap and a fastener, e.g., a screw. The cap can be placed on a proximal end of the button, while the fastener can be inserted through both the cap and the button and into the second bone. The locking assembly can lock the flexible segment at a certain length when the proximal ends of the suture threads are pulled in an approximately proximal direction and/or an approximately perpendicular direction through the one or more second openings and between the proximal end of the button and a distal end of the cap.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example system for semi-rigid bone fixation according to aspects of the present invention.

FIG. 2A is a perspective view of an example apparatus used for semi-rigid bone fixation between two bones according to aspects of the present invention.

FIG. 2B is a perspective view of an example apparatus used for semi-rigid bone fixation between two bones according to aspects of the present invention.

FIG. 3 is a cross-sectional view of the example apparatus of FIG. 2A as installed in a tibia and fibula according to aspects of the present invention.

FIGS. 4A-4B are illustrations of an example locking assembly used in a semi-rigid bone fixation apparatus according to aspects of the present invention.

FIG. 5 is an illustration of an example apparatus used for semi-rigid bone fixation including the example locking assembly of FIGS. 4A-4B according to aspects of the present invention.

FIGS. 6A-6C are illustrations of an example locking assembly used in a semi-rigid bone fixation apparatus according to aspects of the present invention.

FIG. 7 is an illustration of an example apparatus used for semi-rigid bone fixation including the example locking assembly of FIGS. 6A-6C according to aspects of the present invention.

FIGS. 8A-8C are illustrations of an example locking assembly used in a semi-rigid bone fixation apparatus according to aspects of the present invention.

FIG. 9 is an illustration of an example apparatus used for semi-rigid bone fixation including the example locking assembly of FIGS. 8A-8C according to aspects of the present invention.

FIG. 10 shows an example of an entire kit, including an apparatus, such as that shown in FIG. 2A, and its delivery tools according to aspects of the present invention.

FIG. 11 is a cross-sectional view of a tibia and fibula showing an example step of an example method for installing an apparatus, such as that shown in FIG. 2A.

FIG. 12 is a cross-sectional view of a tibia and fibula showing an example step of an example method for installing an apparatus, such as that shown in FIG. 2A.

FIG. 13 is a cross-sectional view of a tibia and fibula showing an example step of an example method for installing an apparatus, such as that shown in FIG. 2A.

FIG. 14 is a cross-sectional view of a tibia and fibula showing an example step of an example method for installing an apparatus, such as that shown in FIG. 2A.

FIGS. 15A-15B are cross-sectional views of a tibia and fibula showing example steps of an example method for installing an apparatus, such as that shown in FIG. 2A.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

The example devices and methods of treatment described herein generally involve providing semi-rigid fixation of two bones, such as the tibia and fibula bones, by implanting an apparatus, including a first anchor, a locking assembly, and a flexible segment, through the fibula bone and into only one side of the tibia bone. That is, the disclosed example devices and methods of treatment do not require a second hole be made through the medial wall of the tibia bone. Instead, an apparatus can be inserted through the fibula bone and only partially into the tibia bone. The disclosed example devices and methods of treatment thus prevent an additional point of necessary patient recovery.

Various example systems and methods are presented herein. Features from each example are combinable with other examples as understood by persons skilled in the pertinent art.

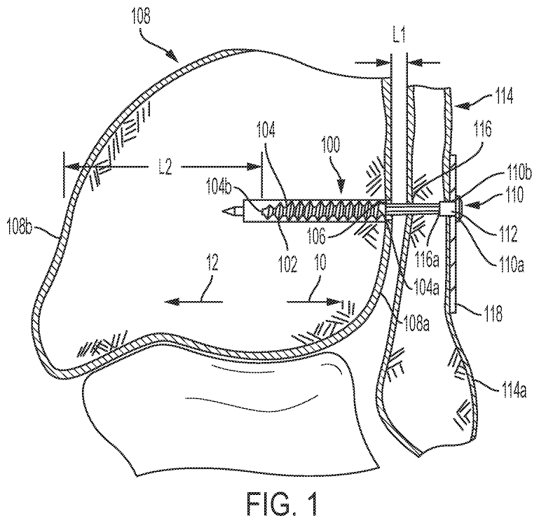

FIG. 1 is an illustration of an example system 100 for semi-rigid bone fixation. The system 100 includes an apparatus 102 for the approximation of two bones, such as the tibia and fibula bones. The apparatus 102 generally includes a first anchor 104, which includes a proximal end 104a and a distal end 104b configured for insertion into a first hole 106 in a first bone 108, e.g., a tibia; a locking assembly 110, which includes a proximal end 110a and a distal end 110b configured for insertion into a second hole 112 in a second bone 114, e.g., a fibula; and a flexible segment 116 extending between the first anchor 104 and the locking assembly 110. The flexible segment 116 is configured to adjust a distance L1 between the first and second bones 108, 114, and the locking assembly 110 releasably engages the flexible segment 116 such that the distance L1 can be adjusted and fixed between the first and second bones 108, 114.

The first anchor 104 can include any type of suture anchor, and can be manufactured from a surgical stainless steel or other suitable biocompatible material, such as 316 LVM stainless steel, titanium, and other suitable materials, such as nitinol, bio-absorbables, or non-absorbables (e.g., PEEK). First anchor 104 can also include an “all-textile” anchor (e.g., VERSALOOP™). The first anchor 104 can pass through the second hole 112 in the second bone 114 and be inserted (e.g., screwed, threaded, or pushed) within the first bone 108 in the first hole 106. The first anchor 104 can be inserted into the first bone 108 from its distal end 104b to its proximal end 104a such that the first anchor 104 does not extend all the way through the first bone 108. That is, the first hole 106 is disposed on a first side 108a (e.g., the lateral or proximal 10 side) of the first bone 108, and the first anchor 104 is configured such that a distance L2 remains between the distal end 104b of the first anchor 104 and a second side 108b (e.g., the medial or distal 12 side) of the first bone 108. In this way, no through or bore hole is required to be made in the second side 108b of the first bone 108.

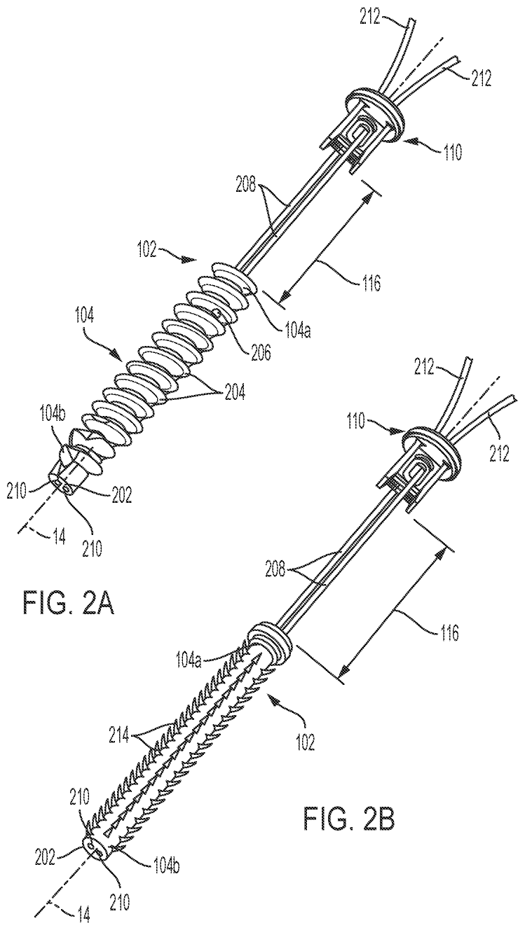

The flexible segment 116 can be manufactured out of a variety of fibers or filaments including but not limited to polymer filaments (e.g. HMWPE, UHMWPE, PET, PTFE, PEEK, PEKK, PLA, PLLA, etc.), metallic filaments (e.g. Nitinol, Titanium, Titanium alloys, Tantalum, Stainless Steel, etc.), or organic filaments (e.g. Collagen, Silk, etc.), or other filaments such as carbon fiber or carbon nanotubes, etc., and can be made of resorbable and/or biologic materials. Flexible segment 116 can include, but is not limited to, a coreless suture, a suture with a jacket and a central core, a tape, or any other tension member available or contemplated, can be poly-coated or uncoated, and can include collagen. Flexible segment 116, which can include one or more suture threads 208 (FIGS. 2A-2B), can extend between the distal end 110b of the locking assembly 110 and the proximal end 104a of the first anchor 104 to adjust the distance L1 between the first and second bones 108, 114. Additionally, the flexible segment 116 can extend beyond the distal end 110b of the locking assembly 110 (e.g., suture threads 208 can be manipulated proximally to the second bone 114), and/or beyond the proximal end 104a of the first anchor 104 (e.g., the suture threads 208 may extend through the first anchor 104 toward its distal tip 202 (FIGS. 2A-2B)), as further described below.

The locking assembly 110 can be manufactured from a surgical stainless steel or other suitable biocompatible material, such as 316 LVM stainless steel, titanium, other suitable materials, such as nitinol, bio-absorbables, or non-absorbables (e.g., PEEK). Locking assembly 110 can also include an “all-textile” anchor (e.g., VERSALOOP™). Locking assembly 110 can be coupled to a bone plate 118 which itself can be coupled to a first side 114a of the second bone 114. As those having skill in the pertinent art will appreciate, bone plate 118 may include one or more holes through which a physician may deliver the apparatus 102 for approximation of the first and second bones 108, 114, as further described below.

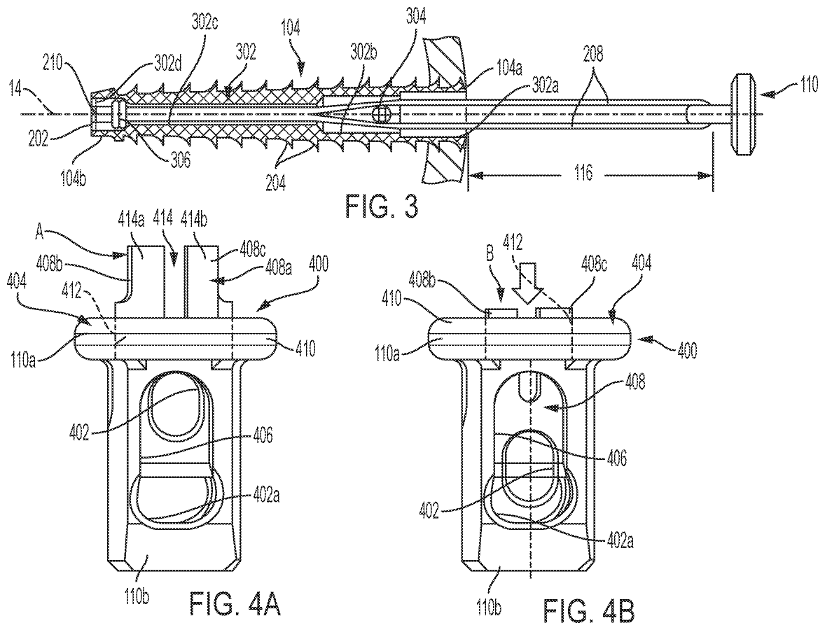

FIGS. 2A-2B and 3 illustrate an example apparatus 102 used to provide semi-rigid bone fixation of the tibia and fibula bones. The first anchor 104 may be a rigid elongate member including proximal end 104a and distal end 104b configured for insertion into the first bone 108 and terminating at a distal tip 202. The first anchor 104 can further include one or more external threads 204 (FIG. 2A) or one or more barbed ribs 214 (FIG. 2B) extending at least partially between the proximal and distal ends 104a, 104b. A first anchor 104 including external threads 204 (FIG. 2A) can be inserted into the first bone 108 via screwing or threading, as further described below. Alternatively, a first anchor 104 including barbed ribs 214 (FIG. 2B) can be inserted into the first bone 108 via pushing. That is, similar to a standard push-in rivet, barbed ribs 214 may be flexible and configured such that when first anchor 104 is pushed into first bone 108, barbed ribs 214 can deflect and then spring back to lock first anchor 104 securely in place within the first bone 108. One of ordinary skill in the pertinent art will appreciate that, aside from the disclosed examples, the first anchor 104 may include any style of bone anchor (e.g., barbed, threaded, screw-in, expanding, interference fit, etc.).

The first anchor 104 can further include a bore 302 (FIG. 3) extending from the proximal end 104a at least partially towards the distal end 104b, generally along a longitudinal axis 14 of the apparatus 102. As particularly shown in FIG. 3, the bore 302 can include a proximal region 302a, an intermediate region 302b distal to the proximate region 302a including a first support structure 304 therein, and a distal region 302c extending from the intermediate region 302b to a recess 302d in the distal tip 202. The intermediate region 302b and distal region 302c may have a circular or other desired cross-sectional shape, with the distal region 302c having a diameter or other maximum cross-section smaller than the intermediate region 302b. The recess 302d may have a diameter or other cross-section larger than the distal region 302c, e.g., to receive a knot 306 or otherwise fixed distal ends 210 (e.g., a crimp eyelet pin, etc.) of suture threads 208 of the flexible segment 116, as described elsewhere herein.

A first support structure 304 may be provided within the bore 302, e.g., across the intermediate region 302b substantially perpendicular to the longitudinal axis 14. In one example apparatus, holes 206 (FIG. 2A) may be provided through opposite side walls of the first anchor 104 into the intermediate region 302b and a first support structure 304, e.g., a pin, may be inserted into the holes 206 such that the first support structure 304 extends across the intermediate region 302b and substantially permanently attached thereto, e.g., by one or more of press-fit or other interference fit, bonding with adhesive, sonic welding, soldering, and the like. In an alternative example apparatus, the holes 206 may be omitted and a first support structure 304 may be inserted through the intermediate region 302b and positioned and fixed across the intermediate region 302b, e.g., by one or more of interference fit, bonding with adhesive, sonic welding, soldering, and the like. In another example apparatus, a support structure may be integrally formed with the first anchor 104, e.g., machined, cast, molded, and the like from the same piece of material as the rest of the first anchor 104. The pin or other first support structure 304 generally has a diameter or other cross-section smaller than the intermediate region 302b such that the flexible segment 116 may be wrapped at least partially around the first support structure 304, as described further elsewhere herein.

The external threads 204 (FIG. 2A) may extend from the proximal end 104a helically towards the distal end 104b, e.g., entirely to the distal tip 202 or the external threads 204 may terminate before the distal tip 202, e.g., to provide a smooth-walled, unthreaded distal tip (not shown). The external threads 204 may have a substantially uniform configuration along the threaded region of the first anchor 104 or the external threads 204 may be varied as desired, e.g., having different heights, edges, and/or axial spacing (threads per millimeter), as desired.

Similar to external threads 204 (FIG. 2A), the barbed ribs 214 (FIG. 2B) may extend from the proximal end 104a toward the distal end 104b, but in a repeating distally downward angled pattern. The barbed ribs 214 may extend from the proximal end 104a entirely to the distal tip 202 or the barbed ribs 214 may terminate before the distal tip 202, e.g., to provide a smooth-walled distal tip (not shown). The barbed ribs 214 may have a substantially uniform configuration along the barbed region of the first anchor 104 or the barbed ribs 214 may be varied as desired, e.g., having different lengths, angles, and/or spacing (barbs per millimeter), as desired.

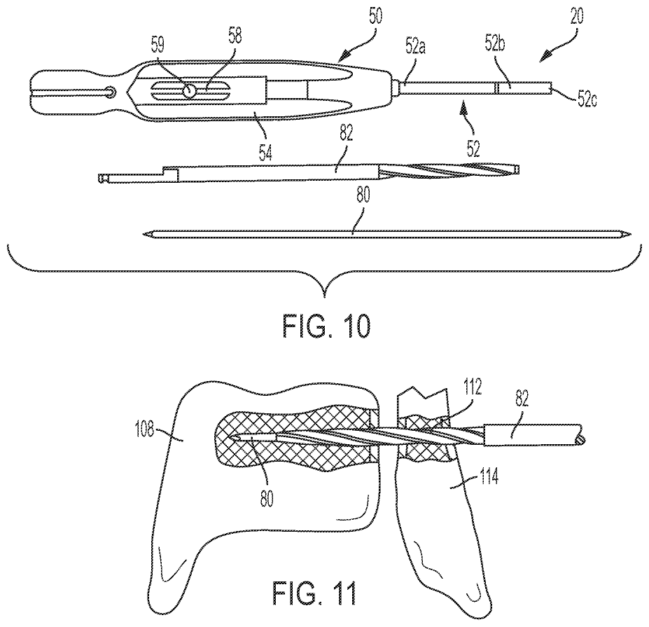

Optionally, as can be seen in FIG. 3, the external threads 204 (or barbed ribs 214) may end before the proximal-most edge of the first anchor 104, e.g., about half to one millimeter (0.5-1.0 mm). Such an offset may facilitate identifying the end of the first anchor 104, e.g., to identify the interface between the first anchor 104 and the first driver tool 50 (not shown, see FIG. 10). In addition, the offset may provide an unthreaded (or un-barbed) region on the proximal end 104a in case the first anchor 104 extends a small distance from a bone into which it is implanted, which may reduce risk of irritation and/or damage to adjacent tissue.

The flexible segment 116 may be an elongate length of suture or other filament having one or more distal ends 210. During assembly, the distal ends 210 may be directed into the bore 302 of the first anchor 104, e.g., through the proximal region 302a, into the intermediate region 302b, wrapped at least partially around the first support structure 304, and then into the distal region 302c until the distal ends 210 exit the bore 302 at the distal tip 202. The distal ends 210 may then be secured together (e.g., by tying one or more knots 306, attaching a crimp eyelet pin, etc.), seated within the recess 302d, that have a cross-section larger than the distal region 302c, thereby preventing the distal ends 210 from being pulled back through the bore 302 during implantation. Alternatively, the distal ends 210 may be directed through the bore 302 from the distal tip 202, wrapping the ends 210 at least partially around the first support structure 304, and exiting the proximal region 302a before tying the knot 306 within the recess 302d. In either of the above-described methods of assembly, one or more proximal ends 212 of suture threads 208 of flexible segment 116 (FIGS. 2A-2B) can be received through the locking assembly 110, as further described below.

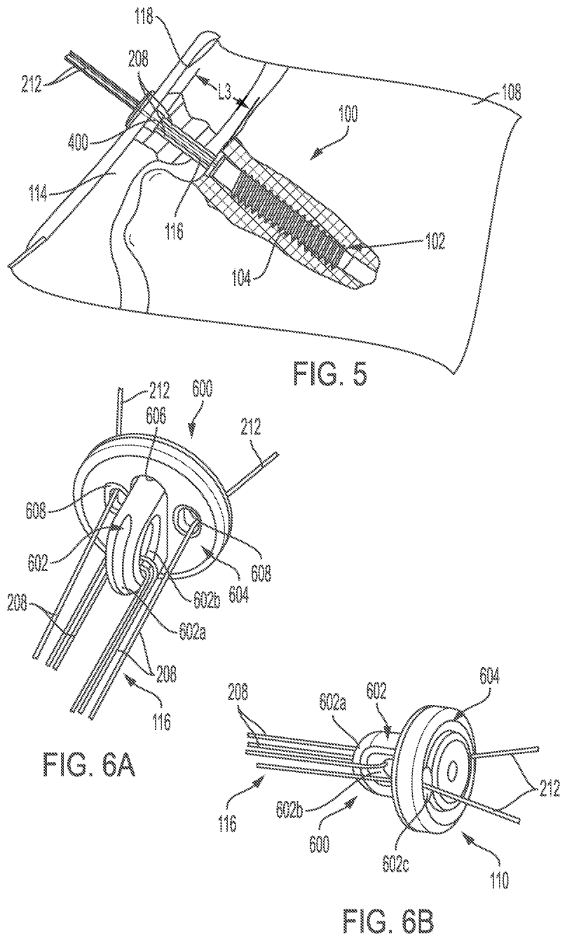

FIGS. 4A-4B and 5 provide an illustration of an example locking assembly 400 that can be used in apparatus 102. Locking assembly 400 can be configured to anchor a proximal end 116a (FIG. 1) of flexible segment 116 to the first side 114a of the second bone 114. To do so, locking assembly 400 includes one or more openings 402 for receiving the proximal ends 212 (FIGS. 2A-2B) of flexible segment 116. Locking assembly 400 can include a body 404 and a locking insert 408. The body 404 can include proximal end 110a, distal end 110b, and an internal cavity 406. The body 404 can have any suitable shape, such as, for example, a cylindrical shape, a rectangular shape, a pyramidal shape, and/or any other suitable shape. The body can further include a proximal cap 410 that can be integral with body 404 or provided as a separate component or piece, connectively attached or received by the body 404. As shown in FIGS. 4A-4B, the proximal cap 410 can be wider, or have a larger diameter, than the distal end 110b of the body 404.

The internal cavity 406 of body 404 can be sized and configured to receive the locking insert 408 therein. In some examples, the internal cavity 406 can extend along a length of the body 404, from the proximal end 110a to the distal end 110b. The proximal end 110a and/or the distal end 110b can define a closed end or an open end. For example, a proximal opening 412 of body 404 can be defined by and extend through the proximal cap 410 to the internal cavity 406 such that the internal cavity 406 defines a channel extending from the proximal opening 412 to the distal end 110b of the body 404. The locking insert 408 can be slidably received within the internal cavity 406 by inserting the locking insert 408 through the proximal opening 412. In some examples, where the body 404 includes a separate proximal cap 410, the locking insert 408 can be inserted into the internal cavity 406 prior to coupling the proximal cap 410 to the body 404. The proximal opening 412 may be a longitudinal opening configured to match a cross-sectional area of the internal cavity 406 and/or a cross-sectional area of the locking insert 408.

In some examples, the locking insert 408 can be slidably moveable within the internal cavity 406 from a first (proximal) position A defining a suture receiving space 402a, as illustrated in FIG. 4A, to a second (distal) position B in which the locking insert 408 is positioned substantially within the suture receiving space 402a, as illustrated in FIG. 4B. As further described below with respect to FIG. 5, transitioning from the first position A to the second position B can lock the flexible segment 116 at length L3. In some examples, the locking insert 408 can include one or more first locking features 408b, 408c sized and configured to couple to and/or interact with one or more second locking features (not illustrated) internal to body 404. The first locking features 408b, 408c (such as tabs extending laterally from the locking insert 408) can interact with a stop surface (not illustrated) internal to body 404 when the locking insert 408 is positioned within the internal cavity 406. The stop surface can be defined by the proximal cap 410 and/or the proximal end 110a of the body 404. The first locking features 408b, 408c can interface with the stop surface which prevents the locking insert 408 from being removed proximally from the internal cavity 406. In some examples, the first locking features 408b, 408c can define a flat proximal surface configured to interface with stop surface. In other examples, the first and second locking features 408b, 408c can include any suitable cooperating locking features.

In some examples, the locking insert 408 can be configured to flex or otherwise deform during insertion into and/or removal from the internal cavity 406. For example, a slot 414 can divide a proximal end 408a of locking insert 408 into a first prong 414a and a second prong 414b that can be flexed towards a center line of the locking insert 408 during insertion and/or removal of the locking insert 408.

In some examples, the body 404 can comprise a material which enables the locking insert 408 to transition from the second (closed) position B to the first (open or unlocked) position A. For example, the body 404 can be temporarily deformable to release the locking insert 408 and/or deform the proximal opening 412 such that the locking insert 408 can be transitioned from the second position B to the first position A. The body 404 can be formed from a semi-resilient material and/or semi-deformable material. Deformation of the body 404 (e.g., compressing or squeezing) releases the first locking features 408b, 408c from the second locking features, allowing the locking insert 408 to transition from the second position B to the first position A. In some examples, deformation of the body 404 further allows the locking insert 408 to be removed from the internal cavity 408. Although examples are discussed herein including a deformable body 404, it will be appreciated that the locking insert 408 can transition from the second position B to the first position A using any suitable system.

The locking assembly 400, as shown in FIGS. 4A-4B and 5, can be configured such that proximal ends 212 of suture threads 208 can extend proximally from locking assembly 400, e.g., through one or more adjustment holes (not illustrated) defined by proximal cap 410. In some examples, the locking insert 408 can be configured to automatically transition from first position A to second position B when the proximal ends 212 are shortened (e.g., tightened) to thereby configure flexible segment 116 at a desired length L3. For example, as shown in FIG. 5, the flexible segment 116 can extend from the locking assembly 400 coupled to the first side 114a of the second bone 114 to the first anchor 104 inserted into the first bone 108. When the proximal ends 212 are shortened, the position of the first and second bones 108, 114 can be adjusted by a force applied by the flexible segment 116. When the force applied to the first and second bones 108, 114 by the flexible segment 116 reaches a certain value, the flexible segment 116 can cause the locking insert 408 to transition from the first position A to the second position B and lock the flexible segment 116 at the desired length L3.

In some embodiments, the locking insert 408 can be transitioned from the first position A to the second position B manually, such as, for example, using a finger, a tool, and/or any other suitable instrument. For example, when the flexible segment 116 is shortened to a desired length L3, a force can be applied to the proximal end 408a of the locking insert 408 to transition the locking insert 408 to the second position B. The transition force causes the first locking features 408b, 408c to interact with the second locking features to lock the flexible segment 116 at the desired length L3.

FIG. 5 is an illustration of an example apparatus 102 used for semi-rigid bone fixation including the example locking assembly 400 of FIGS. 4A-4B. As shown, the flexible segment 116 can be configured such that the first anchor 104 and the locking assembly 400 can be separated by length L3. As described herein, the first anchor 104, with suture threads 208 already attached, can first be inserted through the second bone 114 and into the first bone 108. As such, the proximal ends 212 of suture threads 208 can remain loose and extend proximally outward from the second bone 114. As described above, the proximal ends 212 can then be fed through the openings 402 of locking assembly 400 and/or through the one or more adjustment holes defined by proximal cap 410, and the locking assembly 400 can be inserted into the second bone 114. The proximal ends 212 can then be manipulated (e.g., tightened or loosened) to fix the desired length L3 between the first anchor 104 and the locking assembly 400.

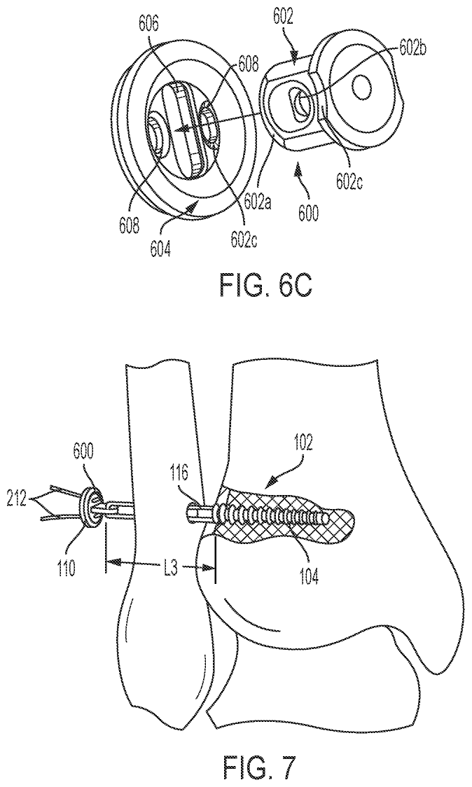

FIGS. 6A-6C and 7 provide an illustration of another example locking assembly 600 that can be used in apparatus 102. Locking assembly 600 can also be configured to anchor the proximal end 116a (FIG. 1) of flexible segment 116 to the first side 114a of the second bone 114. To do so, locking assembly 600 can include a locking insert 602 and a button 604. The button 604 can include a first opening 606 configured to receive a distal end 602a of the locking insert 602, as particularly shown in FIG. 6C. The button 602 can further include one or more second openings 608 configured to accept the proximal ends 212 of the suture threads 208, as particularly shown in FIG. 6A. The locking insert 602 can also include a loop opening 602b configured to receive one or more loops of the suture threads 208 of the flexible segment 116. Locking insert 602 can also include one or more indentations 602c to allow the proximal ends 212 of suture threads 208 to more easily pass through the second openings 608 of the button 604 and then through the indentations 602c of locking insert 602, as particularly shown in FIG. 6B.

In a similar fashion as discussed above with respect to locking assembly 400, locking assembly 600 can be configured such that putting force on the proximal ends 212 of suture threads 208, such as pulling the proximal ends 212 in a proximal direction 10, can force the locking insert 602 to be inserted into and lock inside button 604. The proximal ends 212 can then be manipulated (e.g., tightened or loosened) to fix the desired length L3 between the first anchor 104 and the locking assembly 600, as shown in FIG. 7.

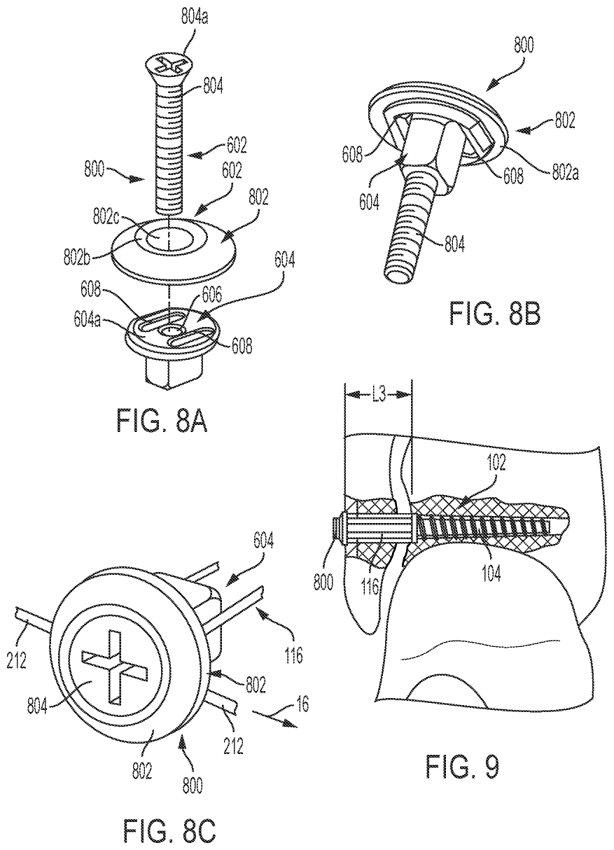

FIGS. 8A-8C and 9 provide an illustration of another example locking assembly 800 that can be used in apparatus 102. Locking assembly 800 can also be configured to anchor the proximal end 116a (FIG. 1) of flexible segment 116 to the first side 114a of the second bone 114. To do so, locking assembly 800 can include locking insert 602 and button 604, as described above with respect to locking assembly 600. With locking assembly 800, however, locking insert 602 may further include a cap 802 configured to be placed on a proximal end 604a of the button 604, and a fastener 804, such as a screw, configured to be screwed or inserted through the cap 802 and the button 604 and into the second bone 114.

The screw 804 is of sufficient strength to withstand the loads of tensioning the locking assembly 800 and to endure subsequent stresses after implantation. Screw 804 can include a flat head 804a to allow screw 804 to lie flush with the cap 802, and additionally can include a cross feature to allow for a standard matching driver. The length and arrangement (e.g., number of threads per centimeter) of the screw threads is sufficient to withstand the required loads and provide adjustment of the locking assembly 804 during installation. As particularly shown in FIG. 8A, the cap 802 can include a countersink feature 802b for receiving the flat head 804a of the screw 804, as well as an undersized through hole 802c to keep the screw 804 captive. The cap 802 can further include a round body with smooth radii edges to aid in concealing the locking assembly 800 beneath the patient's skin. An internal counter bore (not illustrated) can provide clearance for the proximal end 604a of button 604, as well as a load bearing surface for the flexible segment 116. An internal edge radius assists in tensioning the flexible segment 116 and an additional contact surface traps the flexible segment 116 between the cap 802 edge and the second bone 114.

Locking assembly 800 can be configured such that putting force on the proximal ends 212 of suture threads 208, such as pulling the proximal ends 212 in an approximately proximal direction 10 and/or an approximately perpendicular direction 16, allows the proximal ends 212 to be manipulated (e.g., tightened or loosened) to fix the desired length L3 between the first anchor 104 and the locking assembly 800, as particularly shown in FIGS. 8C and 9. Additionally, locking assembly 800 can be configured such that the proximal ends 212 can be pulled as described above and further between the proximal end 604a of the button 604 and a distal end 802a of the cap 802, as particularly shown in FIG. 8C. Once any desired adjustments have been made, locking insert 602 can be tightened down onto and into button 604 and into the second bone 114.

Turing to FIG. 10, an example of an entire kit is shown that may be included in a system 20 for performing a procedure including implantation of an apparatus 102, such as that shown in FIG. 2A. As shown, the kit generally includes a Kirschner wire 80, a custom drill 82 for site preparation, and the first driver tool 50, which houses many of the other components during the early phases of installation. Optionally, the first anchor 104 may be preloaded into the first driver tool 50, as further discussed below.

The first driver tool 50 generally includes an elongate tubular outer shaft 52 including a proximal end 52a having a handle 54 thereon, a distal end 52b terminating in a distal tip 52c, and a lumen (not shown) extending between the proximal and distal ends 52a, 52b. Optionally, the handle 54 includes one or more additional features, e.g., a cleating structure 58 and an actuator 59 for releasably securing the suture threads 208 of flexible segment 116 used to secure the first anchor 104 to the first driver tool 50, as described elsewhere herein.

FIGS. 11-15B provide an example method for installing the apparatus 102, such as that shown in FIG. 2A, between a first bone 108, e.g., the tibia, and the second bone 114, e.g., the fibula, using the system 20 shown in FIG. 10, to provide semi-rigid fixation of the bones relative to one another, e.g., to treat a syndesmotic injury. It will be appreciated that the apparatus, systems, and methods described herein may also be used in other locations and/or procedures, e.g., to provide approximation between two bones other than the tibia and fibula.

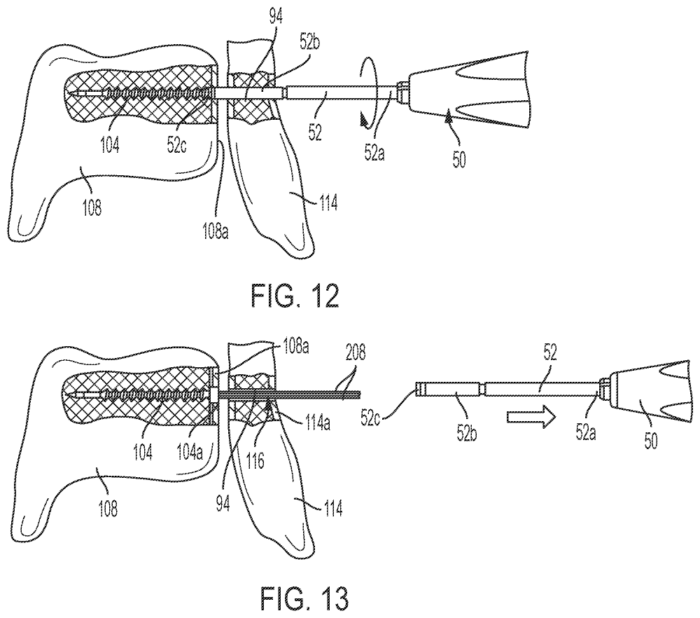

Initially, as shown in FIG. 11, the Kirschner wire 80 may be placed through the second bone 114 and into the first bone 108 at the appropriate location, e.g., using conventional methods, and then a drill 82 may be introduced over the Kirschner wire 80 to create the second hole 112 through the second bone 114 and at least partially into the first bone 108. In the example kit shown in FIG. 10, the drill 82 may use a custom drill bit 82 sized to create a clearance hole 94 (FIG. 12) larger than four millimeters (4.0 mm), e.g., about 4.1 mm, to accommodate a first anchor 104 having an outer thread diameter of four millimeters (4.0 mm). It will be appreciated that other sizes may be provided, as desired.

Turning to FIG. 12, the first anchor 104 may then be introduced through the clearance hole 94 and threaded (or pushed if using first anchor 104 as in FIG. 2B) into the first bone 108 to a desired depth. For example, as described elsewhere herein, the first anchor 104 may be secured to the distal end 52b of the outer shaft 52 of the first driver tool 50. Once the distal tip 202 of the first anchor 104 engages the first bone 108, the first driver tool 50 may be rotated and advanced to thread the first anchor 104 into the first bone 108 to a desired depth, e.g., such that the proximal end 104a of the first anchor 104 is substantially flush with the first side 108a of the first bone 108. As can be seen, the clearance hole 94 drilled through the second bone 114 has sufficient size to accommodate the outer shaft 52 of the first anchor driver tool passing therethrough to thread the first anchor 104 into the first bone 108.

Turning to FIG. 13, once the first anchor 104 is threaded into the first bone 108 to the desired depth, the first driver tool 50 may be removed. Since the first anchor 104 is secured to the outer shaft 52, the user first releases the first anchor 104 from the first driver tool 50, e.g., by releasing the suture threads 208 of the flexible segment 116 from the cleat 58, and then the first driver tool 50 may be withdrawn. As the first driver tool 50 is withdrawn, the proximal ends 212 of the suture threads 208 of the flexible segment 216 may slide through the lumen of the first driver tool 50 and hang freely out the first side 114a of the second bone 114.

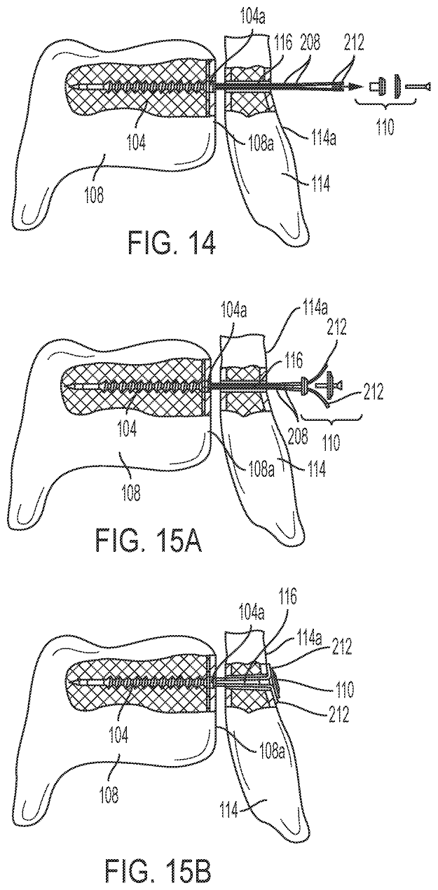

Turning to FIG. 14, once the first driver tool 50 has been removed, proximal ends 212 of the suture threads 208 can be slid through a locking assembly 110 (e.g., 400, 600, 800), as described herein.

Once the proximal ends 212 have been received through the locking assembly 110, as shown in FIG. 15A, the proximal ends 212 can be manipulated (e.g., tightened, loosened, etc.), as described herein, to adjust the length of flexible segment 116 to a desired length L3 (FIGS. 7, 9), thereby fixing the distance between the first and second bones 108, 114. Finally, as shown in FIG. 15B, the loose proximal ends 212 can be removed, e.g., by trimming.

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. An apparatus for the approximation of two bones, comprising:
a first anchor comprising:
a proximal end; and
a distal end configured for insertion into a first hole in a first bone;
a locking assembly comprising:
a proximal end; and
a distal end configured for insertion into a second hole in a second bone; and
a flexible segment extending between the first anchor and the locking assembly and configured to adjust a distance between the first and second bones;
wherein the locking assembly releasably engages the flexible segment to fix the distance between the first and second bones.

2. The apparatus of claim 1, wherein the first anchor passes through the second hole and is threaded within the first bone in the first hole from the distal end to the proximal end.

3. The apparatus of claim 2, wherein the flexible segment extends at least one of between or beyond the distal end of the locking assembly and the proximal end of the first anchor.

4. The apparatus of claim 2, wherein the first hole is disposed on a first side of the first bone, and wherein the first anchor is configured such that there is a distance between the distal end of the first anchor and a second side of the first bone.

5. The apparatus of claim 1, wherein the locking assembly is configured to be coupled to a bone plate, and wherein the bone plate is configured to be coupled to a first side of the second bone.

6. The apparatus of claim 1, wherein the locking assembly further comprises one or more openings for receiving the flexible segment and is configured to anchor a proximal end of the flexible segment to a first side of the second bone.

7. The apparatus of claim 1, wherein the locking assembly further comprises:
a body comprising the proximal end, the distal end, and an internal cavity; and
a locking insert moveable from a first position to a second position within the internal cavity;
wherein the locking assembly is configured to receive the flexible segment through one or more openings; and
wherein the locking insert is configured to transition from the first position to the second position to lock the flexible segment at a length.

8. The apparatus of claim 1, wherein the locking assembly further comprises:
a locking insert; and
a button comprising:
a first opening configured to accept a distal end of the locking insert; and
one or more second openings configured to accept one or more proximal ends of one or more suture threads;
wherein the flexible segment comprises the one or more suture threads.

9. The apparatus of claim 8, wherein the locking assembly is further configured to lock the flexible segment at a length when the one or more proximal ends of the one or more suture threads are pulled in a proximal direction through the one or more second openings.

10. The apparatus of claim 8, wherein:
the locking insert comprises:
a cap configured to be placed on a proximal end of the button; and
a screw configured to be screwed through the cap and the button and into the second bone; and
the locking assembly is further configured to lock the flexible segment at a length when the one or more proximal ends of the one or more suture threads are pulled in at least one of an approximately proximal direction and an approximately perpendicular direction through the one or more second openings and between the proximal end of the button and a distal end of the cap.

11. An apparatus for the approximation of two bones, comprising:
a first anchor comprising:
a proximal end; and
a distal end configured for insertion into a first hole in a first bone;
wherein the first anchor passes through a second hole of a second bone and is inserted within the first bone in the first hole from the distal end to the proximal end;
a locking assembly comprising:
a proximal end; and
a distal end configured for insertion into the second hole in the second bone; and
a flexible segment extending between the first anchor and the locking assembly and configured to adjust a distance between the first and second bones;
wherein the locking assembly releasably engages the flexible segment to fix the distance between the first and second bones.

12. The apparatus of claim 11, wherein the flexible segment extends at least one of between or beyond the distal end of the locking assembly and the proximal end of the first anchor.

13. The apparatus of claim 11, wherein the first hole is disposed on a first side of the first bone, and wherein the first anchor is configured such that there is a distance between the distal end of the first anchor and a second side of the first bone.

14. The apparatus of claim 11, wherein the locking assembly is configured to be coupled to a bone plate, and wherein the bone plate is configured to be coupled to a first side of the second bone.

15. The apparatus of claim 11, wherein the locking assembly further comprises one or more openings for receiving the flexible segment and is configured to anchor a proximal end of the flexible segment to a first side of the second bone.

16. The apparatus of claim 11, wherein the locking assembly further comprises:
a body comprising the proximal end, the distal end, and an internal cavity; and
a locking insert moveable from a first position to a second position within the internal cavity;
wherein the locking assembly is configured to receive the flexible segment through one or more openings; and
wherein the locking insert is configured to transition from the first position to the second position to lock the flexible segment at a length.

17. The apparatus of claim 11, wherein the locking assembly further comprises:
a locking insert; and
a button comprising:
a first opening configured to accept a distal end of the locking insert; and
one or more second openings configured to accept one or more proximal ends of one or more suture threads;
wherein the flexible segment comprises the one or more suture threads.

18. The apparatus of claim 17, wherein the locking assembly is further configured to lock the flexible segment at a length when the one or more proximal ends of the one or more suture threads are pulled in a proximal direction through the one or more second openings.

19. The apparatus of claim 17, wherein:
the locking insert comprises:
a cap configured to be placed on a proximal end of the button; and
a fastener configured to be inserted through the cap and the button and into the second bone.

20. The apparatus of claim 19, wherein the locking assembly is further configured to lock the flexible segment at a length when the one or more proximal ends of the one or more suture threads are pulled in at least one of an approximately proximal direction and an approximately perpendicular direction through the one or more second openings and between the proximal end of the button and a distal end of the cap.
